(19) United States
(12) Patent Application Publication
(10) Pub. No.: US 2007/0249515 A1
(43) Pub. Date: Oct. 25, 2007

(54) Method for Producing Quaternary Hydroxyalkylammonium Granules

(76) Inventors: Georg Borchers, Bad Nauheim (DE); Andreas Schottstedt, Hofheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/578,735

(22) PCT Filed: Apr. 9, 2005
(86) PCT No.: PCT/EP05/03742

§ 371 Date: Apr. 4, 2007

(30) Foreign Application Priority Data
Apr. 17, 2004 (DE) 10 2004 018 751.7

(51) Int. Cl. C07C 211/00; C08B 7/00; C08B 9/00

(52) U.S. Cl. 510/504; 536/56; 536/58; 564/281

(57) ABSTRACT

The present invention relates a method for producing quaternary hydroxyalkylammonium granules, which is characterized by spraying an aqueous solution or suspension comprising a) a quaternary hydroxyalkylammonium compound b) a carrier material and c) optionally binders and/or other additives into a fluidized-bed apparatus. The fluidized-bed consists of granules that have the same composition as the aqueous solution or suspension sprayed into the apparatus.

The invention relates to a method for producing flowable granules characterized by high contents of quaternary hydroxyalkylammonium compounds with very homogeneous distribution of the individual components, and to their use in detergents and cleaners.

Besides a good conditioning effect on textiles, quaternary ammonium compounds which comprise at least one hydroxyalkyl radical exhibit an improvement in the bleaching power of hydrophobic organic peroxo bleach systems, but also an increase in the effects of dye fixing agents, and good dispersibility, as a result of which deposition of dirt and sediments on the cleaned textiles in the wash liquor (graying) is reduced. Moreover, hydroxyalkyl compounds can be readily formulated with surfactants, in particular with long-chain, anionic surfactants and are thus useful additives in modern detergents and cleaners.

Quaternary hydroxyalkylammonium compounds are available as aqueous solutions in relatively low concentrations and, in this form, are problematic for producing solid detergents and cleaners. Concentrating the aqueous solutions produces highly viscous gels which are difficult to handle. Various methods have been used to offer quaternary hydroxyalkylammonium compounds in granule form.

WO 96/17042 describes granular detergent preparations obtained by mixing aqueous solutions of cationic surfactant, anionic surfactant and anionic polymer, concentrating the aqueous solution to >50% by weight surfactant by evaporation and granulation by mixing with a builder. If appropriate, granulation follows a drying step. In WO 98/53037, cationic surfactants, preferably mixed with sodium silicate, are dried in a spray tower.

WO 01/81528 describes a method of granulating cationic, amphoteric or zwitterionic surfactants without adding an anionic surfactant and with reduced temperature expenditure, where inorganic carrier material is initially introduced into a fluidized bed, and an aqueous surfactant solution is sprayed on in the presence of a drying gas at temperatures from 65 to 200° C. A disadvantage which arises with this method is that uniform surfactant distribution and granule composition is not reliably ensured. Metering fluctuations in the product streams in a continuous process or undesired interruptions in a batch process can lead to nonuniform distribution of the formulation constituents. Moreover, it has proven difficult here to fluidize the usually very finely particulate inorganic carrier materials homogeneously and uniformly at the start of the granulation process, meaning that pregranulation of the carrier material is required as an additional process step.

Accordingly, the object of the present invention was to provide a method which avoids the difficulties mentioned and produces flowable granules with high contents of quaternary hydroxyalkylammonium compounds and high homogeneity within the granules.

The invention provides a method for producing quaternary hydroxyalkylammonium granules which are suitable as additive in detergents and cleaners, in which an aqueous solution or suspension (slurry) which comprises

a) a quaternary hydroxyalkylammonium compound

b) a carrier material and

c) optionally binders and/or further additives

is sprayed into a fluidized-bed apparatus, where the fluidized bed consists of granules of the same composition as the sprayed-in aqueous solution or suspension.

Here, granulation of the solution or suspension and simultaneously also drying takes place in the presence of a stream of air at temperatures of approximately 50 to 200° C., preferably 70 to 150° C., particularly preferably 80 to 120° C. The method according to the invention is thus an inherent granulation of the spray liquid with the germ material in the fluidized bed.

By spraying a solution or preferably a suspension, consisting of components a) and b), optionally also c) in defined amounts by weight, via spray nozzles into the fluidized-bed apparatus in which bed material of the same composition is present, granules are produced which have a very uniform distribution of components a), b) and optionally c) in the individual granule. As a result, an improvement in the quality of the granules is achieved, the resulting particles are more homogeneous, the granule spectrum at the outlet is narrow and the individual particles are approximately spherical.

According to the invention, preference is given to granulating quaternary hydroxyalkylammonium compounds according to the formula I 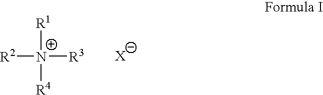
where R¹ is linear or branched C₅-C₂₂-alkyl, C₅-C₂₂-alkenyl, C₅-C₂₂-alkylamidopropyl, C₅-C₂₂-alkenylamidopropyl, C₈-C₂₂-alkoxypropyl or C₅-C₂₂-alkenyloxypropyl, R² may be a C₁-C₂₂-alkyl or C₂-C₂₂-alkenyl group, R³ is C₁-C₂₂-alkyl, C₂-C₂₂-alkenyl or like R⁴ is a group of the formula -A-(OA)ₙOH, where A may be —C₂H₄— and/or —C₃H₆—, and n is a number from 0 to 20 and X is an anion, for example chloride, bromide, iodide, fluoride, sulfate, hydrogen sulfate, carbonate, hydrogen carbonate, phosphate, mono- and dihydrogen phosphate, pyrophosphate, metaphosphate, nitrate, methyl sulfate, phosphonate, methyl phosphonate, methanedisulfonate, methylsulfonate, ethanesulfonate or an anionic compound of the formulae R⁶SO₃^{⊖}, R⁷SO₄^{⊖} or R⁶COO^{⊖}, in which R⁶ and R⁷ are C₈-C₂₀-, preferably C₁₀-C₁₈-alkyl, and R⁷ is additionally also C₇-C₁₈-alkylphenyl.

Preference is given to C₅-C₁₁-alkyldimethylhydroxyethyl quat and C₁₂₋₁₄-alkyldimethylhydroxyethyl quat.

The carrier materials used in the method according to the invention may be of an inorganic and/or organic nature.

Suitable inorganic carriers are finely crystalline zeolite, preferably zeolite A, X and, in particular, zeolite P or mixtures of A, X and/or P. As zeolite P, particular preference is given, for example, to zeolite MAP (Doucil A 24=commercial product from Ineos). The zeolite can be used as a spray-dried powder or in the form of an undried stabilized suspension which is still moist from the preparation. If the zeolite is used as suspension, this can comprise small additives of nonionic surfactants as stabilizers, for example 1 to 3% by weight, based on zeolite.

Besides zeolite, preference is also given to using crystalline sheet silicates, for example SKS 6 from Clariant GmbH and amorphous silicates. Likewise suitable are sodium carbonate, sodium bicarbonate, sodium sulfate, but also alkali metal phosphate, which may be in the form of their alkaline, neutral or acidic sodium or potassium salts. Examples thereof are trisodium phosphate, tetrasodium diphosphate, disodium dihydrogen diphosphate, pentasodium triphosphate, so-called sodium hexametaphosphate, oligomeric trisodium phosphate with degrees of oligomerization of from 5 to 1000, in particular 5 to 50, and mixtures of sodium and potassium salts.

Also of suitability are bentonites, hectorites and saponites, in particular montmorillonites with the formula Naₓ[Al₄₋ₓMgₓ(OH)Si₄O₁₀]*nH₂O where 0.1≦x≦0.4 and 0≦n≦20, preferably x approx. 0.33 and n approx. 4, hectorites with the formula Naₓ[Mg₃₋ₓLiₓSi₄O₁₀]*nH₂O where 0.1≦x≦0.4 and 0≦n≦20, and saponites with the formula Naₓ[Mg₃(Si₄₋ₓAlₓ)₄O₁₀]*nH₂O where 0.1≦x≦0.4 and 0≦n≦20, preferably x approx. 0.33 and n approx. 1. The bentonites, hectorites and saponites can be used as solid or as aqueous dispersions. In general, preference is given to carriers with a more neutral to slightly acidic pH.

Organic carrier materials which can be used are, for example, the carboxylic acids preferably used in the form of their salts, such as citric acid, nitriloacetate (NTA), and ethylenediaminetetraacetic acid, provided such a use is not objectionable on ecological grounds, and phosphonic and polyphosphonic acids. Analogously to this, it is also possible to use polymeric carboxylates and salts thereof. These include, for example, the salts of homopolymeric or copolymeric polyacrylates, polymethacrylates, copolymers of acrylic acid with maleic acid, and polyaspartic acid and also polyvinylpyrrolidone and urethanes. The relative molecular mass of the homopolymers is generally between 1000 and 100 000, that of the copolymers is between 2000 and 200 000, preferably 50 000 to 120 000, based on the free acid, in particular water-soluble polyacrylates are also suitable which are crosslinked, for example, with about 1% of a polyallyl ether of subrose and which have a relative molecular mass above a million. Examples thereof are the polymers obtainable under the names Carbopol 940 and 941.

The granules of components a) quaternary hydroxyalkylammonium compound and b) carriers according to the invention can additionally comprise binders and/or additives.

Suitable binders are cellulose and starch, and ethers or esters thereof, for example carboxymethylcellulose (CMC), methylcellulose (MC) or hydroxyethylcellulose (HEC), methylhydroxyethylcellulose and the corresponding starch derivatives, but also polymeric polycarboxylates, in particular homopolymers or copolymers of acrylic acid, methacrylic acid or maleic acid or salts thereof, also copolymers of these acids with vinyl ethers, such as vinyl methyl ether or vinyl ethyl ether, vinyl esters, such as vinyl acetate or vinyl propionate, acrylamide, methacrylamide, and ethylene, propylene or styrene. Likewise suitable are, in particular, also pulverulent anionic components, in particular alkanesulfonates, alkylarylsulfonates, arylsulfonates, in particular cumene-, xylene-, toluenesulfonate, alkyl ether sulfate, alkyl sulfates, α-olefinsulfonates and soaps. Of particular suitability as binders for the purposes of this invention are sodium carboxymethylcellulose, for example Finnfix BDA from Noviant.

The amount of binders, based on the finished granules, can be 0 to 30% by weight, preferably 1 to 20% by weight.

The granules according to the invention can optionally comprise additives, for example acidifying agents, dispersants, polymers containing sulfonic acid groups, foam inhibitors, chelate complexing agents, bleach activators, bleach catalysts, salts, optical brighteners, graying inhibitors, soil release polymers, dye fixing agents, color transfer inhibitors, preservatives, fragrances and dyes and sequestrants.

Suitable acidifying agents are either inorganic acids, such as, for example, sulfuric acid, sodium hydrogen sulfate, phosphoric acid, sodium hydrogen phosphate, phosphonic acids and salts thereof, or organic acids, in particular mono-, oligo- and polycarboxylic acids or salts thereof, such as citric acid in anhydrous or hydrated form, glycolic acid, succinic acid, succinic anhydride, glutaric acid, glutaric anhydride, adipic acid, adipic anhydride, maleic acid, maleic anhydride or lactic acid, but also acidic polymers. Particularly suitable acidic polymers are polyacrylic acid, polymaleic acid or copolymers of acrylic acid and maleic acid (Sokalan® grades). Acidic additives of this type can also simultaneously have a binder function in the granules.

The amount of acidifying agent is such that the fraction of acidifying agent in the finished granules is approximately 0 to 30% by weight, preferably 1 to 20% by weight.

The use of a dispersant as additive to the spray slurries may be advantageous in order to avoid settling of solids particles. Of suitability are addition products of from 0 to 30 mol of alkylene oxide, in particular ethylene oxide, propylene oxide, butylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan esters; (C₁₂-C₁₈)-fatty acid mono- and diesters of addition products of from 0 to 30 mol of ethylene oxide onto glycerol; glycerol mono- and diesters and sorbitan mono- and diesters of saturated and unsaturated fatty acids having 6 to 22 carbon atoms and optionally their ethylene oxide addition products; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxystearate. Preference is given to liquid fatty acid esters, which may either be ethoxylated (PEG-10 polyglyceryl-2 laurate) or nonethoxylated (polyglyceryl-2 sesquiisostearate).

Further suitable dispersants are sorbitol esters prepared by reacting sorbitol with fatty acid methyl esters or fatty acid triglycerides. The fatty acid radical in the fatty acid methyl esters and fatty acid triglycerides generally comprises 8 to 22 carbon atoms and may be straight-chain or branched, saturated or unsaturated. Examples thereof are palmitic acid, stearic acid, lauric acid, linoleic acid, linolenic acid, isostearic acid or oleic acid. Suitable fatty acid triglycerides are all natural animal or vegetable oils, fats and waxes, for example olive oil, rapeseed oil, palm kernel oil, sunflower oil, coconut oil, linseed oil, castor oil, soybean oil, optionally also in refined or hydrogenated form. Furthermore, anionic dispersants, such as ethoxylated and nonethoxylated mono-, di- or triphosphoric esters, but also cationic dispersants, such as mono-, di- and trialkyl quats and polymeric derivatives thereof can be used.

Mixtures of compounds of two or more of these classes of substance are likewise suitable.

The weight fraction of dispersants in the spray slurries can be 0 to 10% by weight, based on the finished granules.

Particularly preferred polymers containing sulfonic acid groups which can be used are copolymers of unsaturated carboxylic acids, in particular acrylic acid, methacrylic acid and/or maleic acid, monomers containing sulfonic acid groups, for example 1-acrylamido-1-propanesulfonic acid, 2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxypropanesulfonic acid, allylsulfonic acid, methallylsulfonic acid, allyloxybenzenesulfonic acid, methyallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 3-sulfopropyl methacrylate, sulfomethacrylamide, sulfomethylmethacrylamide and optionally further ionic or nonionogenic monomers.

The granules according to the invention can comprise, as foam inhibitors, fatty acid alkyl ester alkoxylates, organopolysiloxanes and mixtures thereof with microfine, optionally silanized silica, and paraffins, waxes, microcrystalline waxes and mixtures thereof with silanized silica. Mixtures of different foam inhibitors can also be used advantageously, e.g. those of silicone oil, paraffin oil or waxes. Preferably, foam inhibitors are bonded to a granular, water-soluble or -dispersible carrier substance.

One further possible group of additives are the chelate complexing agents. Preference is given to polycarboxylic acids, polyamines, for example aminocarboxylates, such as ethylenediamine tetraacetate, N-hydroxyethylethylenediamine triacetate, nitrilotriacetate, ethylenediamine tetrapropionate, triethylenetetraamine hexaacetate, diethylenetriamine pentaacetate, cyclohexanediamine tetraacetate, phosphonates, for example azacycloheptane diphosphonate, Na salt, pyrophosphates, etidronic acid (1-hydroxyethylidene-1,1-diphosphonic acid, 1-hydroxyethane-1,1-diphosphonic acid, acetophosphonic acid) and salts thereof, amino phosphonates, such as ethylenediamine tetrakis(methylenephosphonate), diethylenetriamine pentakis (methylenephosphonate), amine trimethylenephosphonic acid, cyclodextrins, and polyfunctionally substituted aromatic complexing agents, such as dihydroxydisulfobenzene and also ethylenediamine disuccinates. Complexing polymers, i.e. polymers which carry, either in the main chain itself or in lateral positions relative to this, functional groups which can act as ligands and react with suitable metal atoms generally to form chelate complexes, can also be used according to the invention.

Complexing groups of customary complexing polymers are iminodiacetic acid, hydroxyquinoline, thiourea, guanidine, dithiocarbamate, hydroxamic acid, amide oxime, aminophosphoric acid, polyamino, mercapto, 1,3-dicarbonyl radicals.

Base polymers of many complexing polymers are polystyrene, polyacrylates, polyacrylonitriles, polyvinyl alcohols, polyvinylpyridines and polyethyleneimines, and cellulose, starch or chitin.

Bleach activators which are available are N,N,N′,N′-tetraacetylethylenediamine (TAED), nonanoylcaprolactam phenylsulfonate ester (APES), glucose pentaacetate (GPA), xylose tetraacetate (TAX), acyloxybenzenesulfonates (e.g. nonanoyloxybenzenesulfonate (NOBS), sodium 4-benzoyloxybenzenesulfonate (SBOBS), sodium trimethylhexanoyloxybenzenesulfonate (STHOBS)), diacetyldioxohexahydrotriazine (DADHT), tetraacetylglucoluril (TAGU), tetraacetylcyanic acid (TACA), di-N-acetyldimethylglyoxine (ADMG) and 1-phenyl-3-acetylhydantoin (PAH), nitrilotriacetate (NTA) and ammonium nitriles (“nitrile quats”), preferably N,N,N-trimethyl(N-nitrilomethyl)ammonium compounds.

In addition to the bleach activators listed above, or instead of them, the granules according to the invention can also comprise the sulfonimines known from EP 446 982 and EP 453 003 and/or bleach-enhancing transition metal salts or transition metal complexes as so-called bleach catalysts.

In order to bind traces of heavy metals, the salts of polyphosphoric acids, such as 1-hydroxyethane-1,1-diphosphonic acid (HEDP) and diethylenetriaminepentamethylenephosphonic acid (DTPMP) can be used.

Suitable preservatives are, for example, phenoxyethanol, formaldehyde solution, parabens, pentanediol or sorbic acid.

The salts used are, for example, sodium chloride, sodium sulfate, sodium carbonate or sodium silicate.

Optical brighteners which can be used are, for example, compounds from the series of distyrylbenzenes, distyrylbiphenyls, diphenylstilbenes, triazinylaminostilbenes, stilbenzyl-2H-triazoles, for example stilbenzyl-2H-naphthol[1,2-d]triazoles and bis(1,2,3-triazol-2-yl)stilbenes, benzoxazoles, for example stilbenzylbenzoxazole and bis(benzoxazole), furans, benzofurans and benzimidazoles, for example bis(benzo[b]furan-2-yl)biphenyl and cationic benzimidazoles, 1,3-diphenyl-2-pyrazoline, coumarin, naphthalimides, 1,3,5-2-yl derivatives, methine cyanine and dibenzothiophene 5,5-oxide. Preference is given to anionic optical brighteners, in particular sulfonated compounds.

Of suitability here are bis(triazinylamino)stilbenes, preferably sulfonated 4,4′-bis(1,3,5-triazin-2-yl)amino)stilbenes, in particular those of the formula

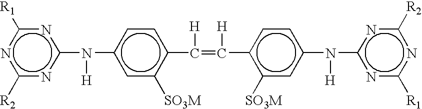

Here, R₁ is a substituted amino group, preferably an anilino, p-sulfoanilino, N-2-hydroxyethyl or NH-2-hydroxyethyl group, R₂ is chlorine, hydroxy, an amino or substituted amino group, for example methylamino, N-2-bis(hydroxyethyl)amino, N-2-hydroxyethyl-N-methylamino, NH-2-methoxyethyl, anilino or morphilino, and M is H or an alkali metal or ammonium ion. The trans form is preferred.

Examples of this type of brightener are 4,4′-bis((4-anilino-6-bis(2-hydroxyethyl)amino-1,3,5-triazin-2-yl)aminostilbene-2,2′-disulfonate, sodium salt, N-2-hydroxyethyl-N-2-methylamino)-1,3,5-triazin-2-yl)amino)stilbene-2,2′-disulfonate, sodium salt (TINOPAL 5BM), 4,4′-bis((4-amino-6-morpholino-1,3,5-triazin-2-yl)amino)stilbene-2,2′-disulfonate, sodium salt (TINOPAL AMS), TINOPAL DCS, where R₁ is p-sulfoanilino and R₂ is N-2-bis(hydroxyethyl)amino, TINOPAL LCS, where R₁ is anilino and R₂ is N-2-methoxyethylamino, TINOPAL TAS, where R₁ and R₂ are anilino and BLANKOPHOR, where R₁ is anilino and R₂ is methylamino.

Further preferably used optical brighteners are distyrylbiphenyls, in particular sulfonated 4,4′-bis(styryl)biphenyls, according to the formula 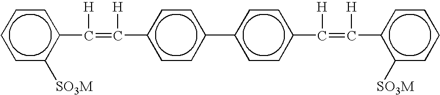
where M is H or alkali metal and the ethenyl groups preferably have a trans configuration. Mention may be made of 4,4′-bis(2-sulfostyryl)biphenyl, sodium salt (TINOPAL CBS).

Also suitable are triazinylaminostilbenes, distyrylbiphenyls and mixtures thereof, 2-(4-styrylphenyl)-2H-naphtho[1,2-d]triazole, 4,4′-bis(1,2,3-triazol-2-yl)stilbene, aminocoumarin, 4-methyl-7-ethylaminocoumarin, 1,2-bis(benzimidazol-2-yl)ethylene, 1,3-diphenylphrazoline, 2,5-bis(benzooxazol-2-yl)thiophenes, 2-styrylnaphtho[1,2-d]oxazole, 2-(4-styryl-3-sulfophenyl)-2H-naphtho[1,2-d]triazole and 2-(stilben-4-yl)-2H-naphthol[1,2-d]triazole.

The content of brighteners in the granules according to the invention is 0.001 to 2% by weight, preferably 0.002 to 0.8% by weight, particularly preferably 0.003 to 0.4% by weight.

Suitable graying inhibitors are carboxymethylcellulose, methylcellulose, hydroxyalkylcellulose, methylhydroxyethylcellulose, methylhydroxypropylcellulose, methylcarboxymethylcellulose and polyvinylpyrrolidone.

Soil release polymers as additives in the granules according to the invention are preferably oligo esters, comprising dicarboxylic acid units and diol units (glycol, alkyl glycol and/or polyol units, in particular polyalkylene polyglycol units. These oligo esters are preferably obtained by polycondensation of one of more aromatic dicarboxylic acids or esters thereof with diols, for example ethylene glycol and/or polyols. If appropriate, these esters can also comprise polyethylene glycol, polypropylene glycol, sulfoisophthalic acid, sulfobenzoic acid, isethionic acid, C₁-C₄-alcohols, oxyalkylated C₁-C₂₄-alcohols, oxyalkylated C₆-C₁₈-alkylphenols and/or oxyalkylated C₈-C₂₄-alkylamines as monomers. Of suitability for producing the oligo esters are, for example, as dicarboxylic acid unit, terephthalic acid, phthalic acid, isophthalic acid, and the mono- and dialkyl esters with C₁-C₆-alcohols, such as dimethyl terephthalate, diethyl terephthalate and di-n-propyl terephthalate, but also oxalic acid, succinic acid, glutaric acid, adipic acid, fumaric acid, maleic acid, itaconic acid, and the mono- and dialkyl esters of the carboxylic acids with C₁-C₆-alcohols, e.g. diethyl oxalate, diethyl succinate, diethyl glutarate, methyl adipate, diethyl adipate, di-n-butyl adipate, ethyl fumarate and dimethyl maleate, and dicarboxylic anhydrides, such as maleic anhydride, phthalic anhydride or succinic anhydride. As polyol unit, preference is given to polyethylene glycols with molar masses of from 500 to 5000, preferably from 1000 to 3000. Furthermore, SRPs comprise, as further component, water-soluble addition products of from 5 to 80 mol of at least one alkylene oxide onto 1 mol of C₁-C₂₄-alcohols, C₆-C₁₈-alkylphenols or C₈-C₂₄-alkylamines. Preference is given to monomethyl ethers of polyethylene glycols.

Suitable alcohols which are alkoxylated are, for example, octyl alcohol, decyl alcohol, lauryl alcohol, myristyl alcohol or stearyl alcohol, but in particular methanol, and the alcohols having 8 to 24 carbon atoms obtainable by the Ziegler process, or the corresponding oxo alcohols. Of the alkylphenols, octylphenol, nonylphenol and dodecylphenol in particular are of importance. Of the suitable alkylamines, the C₁₂-C₁₈-monoalkylamines in particular are used.

Suitable polyols are, for example, pentaerythritol, trimethylolethane, trimethylolpropane, 1,2,3-hexanetriol, sorbitol, mannitol and glycerol.

The invention includes dye fixing agents as additives, for example dye fixing agents which are obtained by reacting diethylenetriamine, dicyandiamide and amidosulfuric acid, amines with epichlorohydrin, for example dimethylaminopropylamine and epichlorohydrin or dimethylamine and epichlorohydrin or dicyandiamide, formaldehyde and ammonium chloride, or dicyandiamide, ethylenediamine and formaldehyde or cyanamide with amines and formaldehyde or polyamines with cyanamides and amidosulfuric acid or cyanamides with aldehydes and ammonium salts, but also polyamine N-oxides, such as, for example, poly(4-vinylpyridine N-oxide), e.g. Chromabond S400, ISP; polyvinylpyrrolidone, e.g. Sokolan HP 50/BASF and copolymers of N-vinylpyrrolidone with N-vinylimidazole and optionally other monomers.

Color transfer inhibitors are also suitable, for example polyamine N-oxides, such as, for example, poly(4-vinylpyridine N-oxide), e.g. Chromabond S-400, ISP; polyvinylpyrrolidone, e.g. Sokalan HP 50/BASF and copolymers of N-vinylpyrrolidone with N-vinylimidazole and optionally other monomers.

Typical individual examples of further additives to be mentioned are sodium borate, starch, sucrose, polydextrose, stilbene compounds, methylcellulose, toluenesulfonate, cumenesulfonate, soaps and silicones.

In one preferred embodiment, an aqueous spray slurry of quaternary hydroxyalkylammonium compound according to formula I, a carrier material and optionally further components produced in a stirred container with stirring using a suitable stirrer at room temperature, optionally heated to a temperature of from 25 to 85° C., is conveyed via a pump to the nozzle and preferably sprayed into the fluidized bed from below.

In this way, it is, for example, possible to granulate a spray slurry which in each case 20 to 60% by weight, preferably 30 to 50% by weight, particularly preferably 35 to 45% by weight, of hydroxyalkyl quat, according to formula I, 40 to 80% by weight, preferably 50 to 70% by weight, particularly preferably 55 to 65% by weight, of carrier material, optionally 0 to 30% by weight, preferably 1 to 20% by weight, of further components according to c), based on the end granules in uncoated form. This process variant, i.e. the introduction of all components in the form of an aqueous spray slurry, can be carried out either in round or rectangular fluidized-bed apparatuses.

If appropriate, solid carrier material can be continuously introduced in addition into the fluidized bed as powdering agent, preferably zeolite, in particular zeolite P, particularly preferably solid material consisting of components a), b) and optionally c). These powdering agents additionally reduce the stickiness of the moist granules during granulation and thus promote the fluidization and drying. The particle size of the powdering agent here is below 100 μm and the granules obtained in this way can comprise 1 to 4% by weight of the powdering agent. For producing granules by the method according to the invention, this variant may be advantageous, although it is not necessarily required. Powdering to reduce the stickiness of the granules can also take place in subsequent work-up steps.

The finished granules are advantageously discharged from the fluidized bed via a size classification of the granules. This classification can take place via a central tube with a countercurrent stream of air (sifter air), which is regulated so that only particles above a certain particle size are discharged from the fluidized bed and smaller particles are retained in the fluidized bed. The particles discharged from the gas stream above the fluidized bed are separated off in a filter or cyclone, any fine dust entrained by the gas can be separated off in a downstream separator, e.g. wet washer. The removed dust is returned to the fluidized bed in the region of the spray nozzle, where renewed wetting takes place.

Granules preferred according to the invention have a d50 value between 0.4 and 2.5 mm. In one particularly preferred embodiment, the granule fraction which is greater than 1.4 mm is returned. This coarse fraction can preferably be added to the fluidized bed either after grinding as a solid component, or it is dissolved again and sprayed into the fluidized bed.

The air inlet temperature here is preferably between 70 and 150° C., preferably between 80 and 120° C. The fluidized air is cooled through heat losses and through the heat of evaporation of the constituents of the solvent. Consequently, the temperature of the bed mass in the fluidized bed is 50 to 80° C., preferably 60 to 75° C. The air exit temperature is preferably between 50 and 70° C.

During the fluidized-bed granulation, the water content of the products can be adjusted within wide limits. In the method according to the invention, drying takes place at the same time as granulation in the fluidized bed. In one preferred embodiment of the invention, the drying leads to the water content of the granules being <2% by weight and the hydroxyethyl quat content being >20% by weight, preferably 30 to 50% by weight, particularly preferably 35 to 45% by weight, based on uncoated granules.

Preferably used round fluidized-bed apparatuses have base plates measuring at least 0.4 m. In particular, preference is given to fluidized-bed apparatuses which have a base plate with a diameter between 0.4 m and 3 m, for example 1.2 m or 2.5 m. In a further preferred embodiment, the fluidized-bed apparatuses can be rectangular in shape. Base plates which may be used are a perforated base plate or a Conidur plate, a wire mesh or a combination base of a perforated plate with a grid.

The granules obtained according to the invention are directly suitable for use in detergents and cleaners. In a particularly preferred use form, however, they can be provided with a coating shell by methods known per se. For this, the granules are coated in an additional step with a film-forming substance, as a result of which the product properties can be influenced considerably. Suitable coating compositions are all film-forming substances, such as waxes, silicones, fatty acids, fatty alcohols, soaps, anionic surfactants, nonionic surfactants, cationic surfactants, anionic and cationic polymers, polyethylene glycols and polyalkylene glycols.

Of suitability are C₈-C₃₁-fatty acids (e.g. lauric acid, myristic acid, stearic acid), dicarboxylic acids, for example glutaric acid, adipic acid or anhydrides thereof; phosphonic acids, optionally phosphonic acids in admixture with other customary coating compositions, in particular fatty acids, for example stearic acid, C₈-C₃₁-fatty alcohols; polyalkylene glycols (e.g. polyethylene glycols with a molar mass of from 1000 to 50 000 g/mol); nonionics (e.g. C₈-C₃₁-fatty alcohol polyalkoxylates having 1 to 100 mol of EO); anionics (e.g. alkanesulfonates, alkylbenzenesulfonates, α-olefin-sulfonates, alkyl sulfates, alkyl ether sulfates with C₈-C₃₁-hydrocarbon radicals); polymers (e.g. polyvinyl alcohols); waxes (e.g. montan waxes, paraffin waxes, ester waxes, polyolefin waxes); silicones.

The coating substance which softens or melts in the range from 30 to 100° C., moreover, can comprise further substances which do not soften or melt within this temperature range, in dissolved or suspended form, e.g.: polymers (e.g. homo-, co- or graft copolymers of unsaturated carboxylic acids and/or sulfonic acids, and alkali metal salts thereof, cellulose ether, starch, starch ether, polyvinylpyrrolidone); organic substances (e.g. mono- or polybasic carboxylic acids, hydroxycarboxylic acids or ether carboxylic acids with 3 to 8 carbon atoms, and salts thereof); dyes; inorganic substances (e.g. silicates, carbonates, bicarbonates, sulfates, phosphates, phosphonates).

Depending on the desired properties of the coated activator granules, the content of coating substance can be 1 to 30% by weight, preferably 5 to 15% by weight, based on coated activator granules.

To apply the coating substances, use may be made of mixers (mechanically induced fluidized bed) and fluidized-bed apparatuses (pneumatically induced fluidized bed). Possible mixers are, for example, plowshare mixers (continuous and batchwise), annular layer mixers and also Schugi mixers. When using a mixer, the heat treatment can take place in a granule prewarmer and/or in the mixer directly and/or in a fluidized bed downstream from the mixer. To cool the coated granules, granule coolers or fluidized-bed coolers can be used. In the case of fluidized-bed apparatuses, the heat treatment takes place via the hot gas used for the fluidizing. The granules coated by the fluidized-bed method can be cooled similarly to the mixer method via a granule cooler or a fluidized-bed cooler. Both in the case of the mixer method and also in the case of the fluidized-bed method, the coating substance can be sprayed on via a single-material or a dual-material nozzle device.

The heat treatment consists in a heat treatment at a temperature of from 30 to 100° C., but the same as or below the melting or softening temperature of the particular coating substance. Preference is given to working at a temperature which is just below the melting or softening temperature.

The precise temperature during the heat treatment or the temperature difference from the melting point of the coating substance is dependent on the coating amount, the heat-treatment time and the properties desired for the coated bleach activator granules and has to be determined for the particular system in preliminary experiments.

Moreover, the granules according to the invention can also comprise further suitable additives such as anionic and nonionic surfactants, which contribute to more rapid dissolution of the granules according to the invention.

Preferred anionic surfactants are alkali metal salts, ammonium salts, amine salts and salts of amino alcohols of the following compounds: alkyl ether sulfates, alkylamide sulfates and ether sulfates, alkylaryl polyether sulfates, monoglyceride sulfates, alkylamide sulfonates, alkyl sulfosuccinates, alkyl ether sulfosuccinates, alkylamide sulfosuccinates, alkyl sulfoacetates, alkylpolyglycerol carboxylates, alkyl phosphates, alkyl ether phosphates, alkyl sarcosinates, alkyl polypeptidates, alkylamidopolypeptidates, alkyl isethionates, alkyl taurates, alkyl polyglycol ether carboxylic acids or fatty acids, such as oleic acid, ricinoleic acid, palmitic acid, stearic acid, copra oil acid salt or hydrogenated copra oil acid salts. The alkyl radical of all of these compounds normally comprises 8-32, preferably 8-22, carbon atoms. Preferred nonionic surfactants are polyethoxylated, polypropoxylated or polyglycerylated ethers of fatty alcohols, polyethoxylated, polypropoxylated and polyglycerylated fatty acid esters, polyethyloxylated esters of fatty acids and of sorbitol, polyethoxylated or polyglycerylated fatty amides.

The granules produced according to the invention are characterized by very good flowability, high active contents, high homogeneity of the components within the granules and by good storage stability in solid washing and cleaning formulations.

Detergents and cleaners comprising the hydroxyalkyl quat granules produced according to the invention can have further customary detergent ingredients, such as surfactants of a nonionic, anionic, cationic or amphoteric nature, organic and inorganic builders and cobuilders, enzymes, bleach activators, bleach catalysts, salts, optical brighteners, graying inhibitors, foam inhibitors, sequestrants, and fragrances and dyes.

Preferred nonionic surfactants are fatty alcohol oxyethylates with about 1 to about 25 mol of ethylene oxide. The alkyl chain of the aliphatic alcohols can be linear or branched, primary or secondary, and generally comprises from 8 to 22 carbon atoms. Particular preference is given to the condensation products of alcohols which comprise an alkyl chain of from 10 to 20 carbon atoms with 2 to 18 mol of ethylene oxide per mole of alcohol. The alkyl chain can be saturated or unsaturated. The alcohol ethoxylates can likewise have a narrow homolog distribution of the ethylene oxide (narrow range ethoxylates) or a broad homolog distribution of the ethylene oxide (broad range ethoxylates). Examples of commercially available nonionic surfactants of this type are Tergitol™ 15-S-9 (condensation product of a C₁₁-C₁₅ linear secondary alcohol with 9 mol of ethylene oxide), Tergitol™ 24-L-NMW (condensation product of a C₁₂-C₁₄-linear primary alcohol with 6 mol of ethylene oxide having a narrow molecular weight distribution). This product class likewise includes the Genapol™ grades from Clariant GmbH.

Moreover, other known types of nonionic surfactants are also suitable, such as polyethylene, polypropylene, polybutylene and polypentylene oxide adducts of fatty alcohols with 8-22 carbon atoms and alkylphenols having 6 to 12 carbon atoms in the alkyl chain, addition products of ethylene oxide with a hydrophobic base formed from the condensation of propylene oxide with propylene glycol or addition products of ethylene oxide with a reaction product of propylene oxide and ethylenediamine.

Furthermore, semipolar nonionic surfactants, for example amine oxides, can be used.

Suitable amine oxides are particularly C₁₀-C₁₈-alkyldimethylamine oxides and C₈-C₁₂-alkoxyethyldihydroxyethylamine oxides.

Suitable anionic surfactants are primarily straight-chain and branched alkyl sulfates, -sulfonates, carboxylates, phosphates, alkyl ester sulfonates, arylalkylsulfonates, alkyl ether sulfates and mixtures of said compounds. The text below will describe some of the suitable types of anionic surfactants in more detail.

Alkyl Ester Sulfonates

Alkyl ester sulfonates represent linear esters of C₈-C₂₀-carboxylic acids (i.e. fatty acids) which are sulfonated by SO₃, as described in “The Journal of the American Oil Chemists Society”, 52 (1975), pp. 323-329. Suitable starting materials are natural fatty derivatives such as, for example, tallow or palm oil fatty acid.

Alkyl Sulfates

Alkyl sulfates are water-soluble salts or acids of the formula ROSO₃M, in which R is preferably a C₁₀-C₂₄-hydrocarbon radial, preferably an alkyl or hydroxyalkyl radial having 10 to 20 carbon atoms, particularly preferably a C₁₂-C₁₈-alkyl or hydroxyalkyl radical. M is hydrogen or a cation, e.g. an alkali metal cation (e.g. sodium, potassium, lithium) or ammonium or substituted ammonium, e.g. a methyl-, dimethyl- and trimethylammonium cation or a quaternary ammonium cation, such as tetramethylammonium and dimethylpiperidinium cation and quaternary ammonium cations, derived from alkylamines such as ethylamine, diethylamine, triethylamine and mixtures thereof. Alkyl chains with C₁₂-C₁₆ are preferred here for low washing temperatures (e.g. below about 50° C.) and alkyl chains with C₁₆-C₁₈ are preferred for higher washing temperatures (e.g. above about 50° C.).

Alkyl Ether Sulfates

The alkyl ether sulfates are water-soluble salts or acids of the formula RO(A)ₘSO₃M, in which R is an unsubstituted C₁₀-C₂₄-alkyl or hydroxyalkyl radical having 10 to 24 carbon atoms, preferably a C₁₂-C₂₀-alkyl or hydroxyalkyl radical, particularly preferably a C₁₂-C₁₈-alkyl or hydroxyalkyl radical. A is an ethoxy or propoxy unit, m is a number greater than 0, typically between about 0.5 and about 6, particularly preferably between about 0.5 and about 3 and M is a hydrogen atom or a cation, such as, for example, a metal cation (e.g. sodium, potassium, lithium, calcium, magnesium, etc.), ammonium or a substituted ammonium cation. Examples of substituted ammonium cations are methyl-, dimethyl-, trimethylammonium and quaternary ammonium cations, such as tetramethylammonium and dimethylpiperidinium cations, and those which are derived from alkylamines, such as ethylamine, diethylamine, triethylamine, mixtures thereof and the like. Examples which may be mentioned are C₁₂-C₁₈-alkyl polyethoxylate (1.0) sulfate, C₁₂-C₁₈-alkyl polyethoxylate (2.25) sulfate, C₁₂-C₁₈-alkyl polyethoxylate (3.0) sulfate, C₁₂-C₁₈-alkyl polyethoxylate (4.0) sulfate, where the cation is sodium or potassium.

Other anionic surfactants which are useful for use in detergents and cleaners are C₈-C₂₄-olefinsulfonates, sulfonated polycarboxylic acids prepared by sulfonation of the pyrolysis products of alkaline earth metal citrates, as described, for example, in the British patent GB 1,082,179, alkyl glycerol sulfates, fatty acyl glycerol sulfates, oleyl glycerol sulfates, alkylphenol ether sulfates, linear or branched alkylbenzenesulfonates, primary and secondary paraffinsulfonates, alkyl phosphates, alkyl ether phosphates, isethionates, such as acyl isethionates, N-acyltaurides, alkyl succinamates, sulfosuccinates, monoesters of the sulfosuccinates (particularly saturated and unsaturated C₁₂-C₁₈-monoesters) and diesters of the sulfosuccinates (particularly saturated and unsaturated C₁₂-C₁₈-diesters), acyl sarcosinates, sulfates of alkylpolysaccharides, such as sulfates of alkylpolyglycosides, branched primary alkyl sulfates and alkyl polyethoxycarboxylates, such as those of the formula RO(CH₂CH₂)ₖCH₂COO⁻M⁺, in which R is a C₈-C₂₂-alkyl, k is a number from 0 to 10 and M is a cation which forms a soluble salt. Resin acids or hydrogenated resin acids, such as rosin or hydrogenated rosin or tall oil resins and tall oil resin acids can likewise be used. Further examples are described in “Surface Active Agents and Detergents” (Vol. I and II, Schwartz, Perry and Berch). A large number of such surfactants are also claimed in the U.S. Pat. No. 3,929,678.

Examples of amphoteric surfactants which can be used in the formulations of the present invention are primarily those which are described as derivatives of aliphatic secondary and tertiary amines in which the aliphatic radical may be linear or branched and in which one of the aliphatic substituents contains between 8 and 18 carbon atoms and comprises an anionic, water-soluble group, such as, for example, carboxy, sulfonate, sulfate, phosphate or phosphonate.

Preferred amphoteric surfactants are monocarboxylates and dicarboxylates, such as cocoamphocarboxypropionate, cocoamidocarboxypropionic acid, cocoamphocarboxyglycinate (or also referred to as cocoamphodiacetate) and cocoamphoacetate.

Further preferred amphoteric surfactants are alkyldimethylbetaines, alkylamidobetaines and alkyldipolyethoxybetaines with an alkyl radical, which may be linear or branched, having 8 to 22 carbon atoms, preferably having 8 to 18 carbon atoms and particularly preferably having 12 to 18 carbon atoms. These compounds are marketed, for example, by Clariant GmbH under the trade name Genagen® CAB and LAB.

Typical examples of cationic surfactants are quaternary ammonium compounds, ester quats, ether quats, ethoxylated quats, in particular quaternized fatty acid alkanolamine ester salts and dialkylamino-propylamine ester salts.

Suitable enzymes are those from the class of proteases, lipases, amylases, pullinases, cutinases, and cellulases and mixtures thereof. Proteases which are available are BLAP®, Opticlean®, Maxacal®, Maxapem®, Esperase®, Savinase®, Purafect®, OxP and/or Duraxym®, amylases which are available are Termamyl®, Amylase-LT®, Maxamyl®, Duramyl® and/or Pruafect® OxAm, and lipases which are available are Lipolase®, Lipomax®, Lumafast® and/or Lipozym®.

The enzymes can be adsorbed to carrier substances and/or embedded in coating substances.

Salts and extenders used are, for example, sodium sulfate, sodium carbonate or sodium silicate (waterglass).

Further constituents of the detergent formulation may be optical brighteners, for example derivatives of diaminostilbenedisulfonic acid or alkali metal salts thereof, and foam inhibitors, such as fatty acid alkyl ester alkoxylates, organopolysiloxanes and mixtures thereof with microfine, optionally silanized silica, and paraffins, waxes, microcrystalline waxes and mixtures thereof with silanized silica. Mixtures of different foam inhibitors can also be used advantageously, e.g. those from silicone oil, paraffin oil or waxes. Preferably, foam inhibitors are bound to a granular carrier substance which is soluble or dispersible in water. In order to bind traces of heavy metals, the salts of polyphosphonic acids, such as 1-hydroxyethane-1,1-diphosphonic acid (HEDP), ethylenediaminetetramethylenephosphonic acid (EDTMP) and diethylenetriaminepentamethylenephosphonic acid (DTPMP), can be added. Typical individual examples of further additives are sodium borate, cellulose and starch, and ethers and esters thereof, sucrose, polydextrose and polymeric additives.

The examples below are intended to illustrate the invention in more detail without limiting it thereto.

Granulation of C_{12/14}-alkyldimethylhydroxyalkylammonium chloride

### EXAMPLE 1

### Laboratory Granulation without Binder

1.1) Preparation of a Spray Slurry:

In the first step, 500 g of a 40% strength, aqueous C_{12/14}-alkyldimethyl-hydroxyethylammonium chloride solution (Präpagen HY from Clariant) were mixed and homogenized with 300 g of zeolite P (Doucil A 24 from Ineos) at room temperature using an Ultra-Turrax®. Within a mixing time of about 5 min, a stirrable and pumpable, homogeneous suspension was prepared which had no recognizable sedimentation, even without stirring. During the mixing operation, a slight temperature increase from 24° C. to about 30-33° C. was established. The suspension prepared in this way had a high solids content of about 62.5% and could be conveyed at room temperature without problems using a peristaltic pump and atomized via a dual-material nozzle.

1.2) Preparation of the Initial Charge Product

In order to be able to operate the granulation process, a fluidized mass is required onto which the active ingredient solution can be sprayed. Specifically at the start of a process development there is generally no suitable fluidized mass yet available, however. For this reason, a batch of the slurry according to example 1.1) was dried in the drying cabinet (about 100° C.) and then worked up. For this, the dried mass was passed through a sieve to give a fine to granular product. For the granulation experiment, therefore, a starting mass (100-250 g) in the granule fraction <1400 μm was present.

1.3) Fluidized-Bed Granulation:

For laboratory experiments, a batchwise operating laboratory fluidized bed of the type GPCG 1.1 from Glatt with an inflow diameter of D=150 mm was used, the spray nozzle spraying into the fluidized bed from below. The dried product as in example 1.2) was initially introduced into the fluidized-bed apparatus. In subsequent experiments, the fines from the preceding granulation experiment in each case were also used. Via the dual-material nozzle, the nonheated slurry was atomized from below onto the mobilized fluidized bed. The amount of spray liquid conveyed using a peristaltic pump was ascertained gravimetrically by means of a balance. The gas volume stream for fluidizing the bed material was about 25 m³/h. The inlet air temperature was adjusted to about 93-95° C. Setting a spray output of about 7-9 g/min of slurry, a temperature in the fluidized bed of about 70-74° C. was established, this temperature level being kept stable. With these operating conditions, an exit air temperature of about 65-69° C. was established. After an experiment run time of about 60 min, clearly recognizable granules had formed. After cooling the granules, fractionation was carried out by sieving off the coarse fractions >1400 μm and the fine fractions <200 μm. With these experiment settings, a granule yield of about 97% was achieved for the target granule range. The bulk density of the granules was about 636 g/l. The composition of the final granules was 40% by weight of C12/14-alkyldimethylammonium chloride and 60% by weight of zeolite P.

### EXAMPLE 2

### Laboratory Granulation with Binder

2.1) Preparation of a Spray Slurry:

In the first step, 1500 g of a 40% strength, aqueous C_{12/14}-alkyldimethyl-hydroxyethylammonium chloride solution (Prapagen HY from Clariant) were mixed and homogenized with 870 g of zeolite P (Doucil A 24 from Ineos), 30 g of tylose as binder (Finnfix BDA from Noviant), and 320 g of water for setting the viscosity at room temperature using an Ultra-Turrax®. Within a mixing time of about 5 min, a stirrable and pumpable, homogeneous suspension was produced, which had no recognizable sedimentation even without stirring. During the mixing operation, a slight temperature increase from 24° C. to about 30-33° C. was established.

The suspension produced in this way had a high solids content of about 55% and could be conveyed at room temperature without problems using a peristaltic pump and be atomized via a dual-material nozzle.

2.2) Preparation of the Initial Charge Product

In order to be able to operate the granulation process, a fluidized mass is required onto which the active ingredient solution can be sprayed. Consequently, a batch of the slurry as in example 2.1) was dried in the drying cabinet (about 100° C.) and then worked up. For this, the dried mass was passed through a sieve to give a fine to granular product. For the granulation experiment, a starting mass (100-250 g) in the granule fraction <1400 μm was thus present.

2.3) Fluidized-Bed Granulation:

For laboratory experiments, a batchwise operating laboratory fluidized bed of type GPCG 1.1 from Glatt with an inflow diameter of D=150 mm was used, the spray nozzle spraying into the fluidized bed from below. The dried product from the drying-cabinet work-up as in example 2.2) was initially introduced into the fluidized-bed apparatus. In subsequent experiments, the fines from the preceding granulation experiment in each case were also used. Via the dual-material nozzle, the nonheated slurry was atomized from below onto the mobilized fluidized bed. The amount of spray liquid conveyed using a peristaltic pump was determined gravimetrically by means of a balance. The gas volume stream for fluidizing the bed material was about 25 m³/h. The inlet air temperature was adjusted to about 94-96° C. Setting a spray output of about 6-6.5 g/min of slurry, a temperature in the fluidized bed of about 72-75° C. was established, this temperature level being kept stable. With these operating conditions, an exit air temperature of about 69-71° C. was established. After an experiment run time of about 198 min, clearly recognizable granules had formed. After cooling the granules, fractionation was carried out by sieving off the coarse fractions >1400 μm and fine fractions <200 μm. With these experiment settings, a granule yield of about 88% was achieved for the target granule range. The bulk density of the granules was about 645 g/l. The composition of the final granules was 40% by weight of C_{12/14}-alkyldimethylammonium chloride and 58% by weight of zeolite P and 2% tylose.

1. A method for producing quaternary hydroxyalkylammonium granules wherein an aqueous solution or suspension having a composition which comprises
20 to 60% by weight of quaternary hydroxyalkylammonium compound,
40 to 80% by weight of carrier material,
0 to 30% by weight of binder
0 to 30% by weight of acidic additive and
0 to 10% by weight of dispersant,
in each case based on said granules, is sprayed into a fluidized-bed apparatus, containing a fluidized-bed of granules having the same composition as the sprayed-in aqueous solution or suspension.

2. The method as claimed in claim 1, wherein the quaternary hydroxyalkylammonium compound used is a compound of the formula I 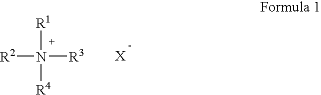
where R¹ is linear or branched C₅-C₂₂-alkyl, C₅-C₂₂-alkenyl, C₅-C₂₂-alkyl-amidopropyl, C₅-C₂₂-alkenylamidopropyl, C₈-C₂₂-alkoxypropyl or C₅-C₂₂-alkenyloxypropyl, R² may be a C₁-C₂₂-alkyl or C₂-C₂₂-alkenyl group, R³ is C₁-C₂₂-alkyl, C₂-C₂₂-alkenyl or like R⁴ is a group of the formula -A-(OA)n—OH, where A may be —C₂H₄— and/or —C₃H₆—, and n is a number from 0 to 20 and X is an anion.

3. The method as claimed in claim 1, wherein the quaternary hydroxylalkylammonium compound used is a compound of the formula I where R¹ is C₅-C₁₁-alkyl or C₁₂-C₁₄-alkyl, R² and R³ are methyl, R⁴ is hydroxyethyl and A is chloride or methosulfate.

4. The method as claimed in claim 1, wherein the carrier material is selected from the group consisting of zeolite, sheet silicate, bentonite, an inorganic salt, a salt of an organic carboxylic acid, and mixtures thereof.

5. The method as claimed in claim 1, wherein the binder is selected from the group consisting of cellulose, starch, an ether or an ester of cellulose, an ether or an ester of starch, a polymeric polycarboxylate, an acidic polymer, and mixtures thereof.

6. The method as claimed in claim 1, wherein the composition further comprises an additive selected from the group consisting of polymers containing sulfonic acid groups, foam inhibitors, chelate complexing agents, bleach activators, bleach catalysts, salts, optical brighteners, graying inhibitors, soil release polymers, dye fixing agents, color transfer inhibitors, preservatives, fragrances, dyes, sequestrants, and mixtures thereof.

7. (canceled)

8. A detergent or cleaner comprising granules produced as in claim 1.

9. The method of claim 1, wherein the aqueous solution or suspension is conveyed into the fluidized-bed apparatus at a point below the fluidized-bed in the presence of a drying gas stream to provide a homogeneous spherical granule having said composition.

10. The method of claim 9, wherein the drying gas stream is air at a temperature of from 50 to 200° C.
